(19) United States
(12) Patent Application Publication
(10) Pub. No.: US 2004/0162479 A1
(43) Pub. Date: Aug. 19, 2004

(54) Percutaneous removal of sentinel lymph node using contrast imaging for identification

(75) Inventors: Ronald Yamamoto, San Francisco, CA (US); Roberta Lee, Redwood City, CA (US); Niyazi Beyhan, Santa Clara, CA (US)

Correspondence Address: Jung-hua Kuo, Attorney At Law, PO Box 3275, Los Altos, CA, 94024, US

(73) Assignee: MANOA MEDICAL, INC., Redwood City, CA

(21) Appl. No.: 10/735,331

(22) Filed: Dec. 12, 2003

Related U.S. Application Data
(60) Provisional application No. 60/433,261, filed on Dec. 12, 2002.

(51) Int. Cl. A61B 5/05

(52) U.S. Cl. 600/411; 600/427

(57) ABSTRACT

A system and method for percutaneous removal of a sentinel lymph node using a radiological contrast agent for identification and guidance of the procedure are disclosed. The method generally includes injecting a radiological contrast agent detectable by an imaging modality into an area of interest, identifying a sentinel lymph node in at least one area of draining lymph nodes that receive lymphatic fluid from the area of interest by imaging the area(s) of draining lymph nodes utilizing the imaging modality, introducing a percutaneous excision device into the area(s) of the draining lymph nodes, and excising the identified sentinel lymph node in the area(s) of draining lymph nodes using the percutaneous excision device, where the introducing and/or the excising is performed under guidance by imaging at least portions of the identified sentinel lymph node and the percutaneous excision device. A second agent may be injected with the radiological contrast agent for increased sensitivity of identification.

### CROSS REFERENCE TO RELATED APPLICATION

This application claims priority to U.S. Provisional Patent Application Serial No. 60/433,261 entitled “System and Method for Percutaneous Removal of Sentinel Lymph Node Using Contrast Imaging for Identification” and filed on Dec. 12, 2002, the entirety of which is incorporated herein by reference.

### BACKGROUND OF THE INVENTION

1. Field of the Invention

The present invention relates generally to minimally invasive procedures. More specifically, a system and method for percutaneous removal of a sentinel lymph node using a radiological contrast agent for identification and guidance of the procedure are disclosed.

2. Description of Related Art

Once cancer develops in one area of the body, cancer cells can travel to other areas of the body via the lymphatic system. Upon leaving an organ or area of soft tissue, the lymphatic vessels drain into a set of lymph nodes. Cancerous cells that are transported within the lymphatic vessel are often entrapped within these lymph nodes. Surgical excision and pathological examination of these draining lymph nodes are important in the clinical staging of cancer. If cancer is not seen in the lymph nodes, then the likelihood of spread to other areas of the body is minimal. If cancerous cells are seen in the lymph nodes, then the possibility of metastatic spread is increased which is often an indication for additional treatment such as chemotherapy.

Removal of all the draining lymph nodes is an invasive surgical procedure which can result in lasting problems. In breast cancer, the draining lymph nodes are often in the ipsilateral axilla. Removal of these lymph nodes may lead to chronic pain, decreased mobility and permanent swelling of the arm. With increasing use of screening mammography, smaller breast cancers are being diagnosed. These early stage breast cancers are often still confined to the breast and have not spread via the lymphatics resulting in negative lymph node dissections. As a result, axillary dissection to remove the lymph nodes often leads to more problems with little benefit to the patient. A less invasive procedure to determine whether the cancer has spread to the draining lymph nodes was introduced (Giuliano A E et al., Ann Surg 1994; 220:391-401). The sentinel lymph node procedure for the breast was adopted from similar sentinel lymph node identification for melanoma. In melanoma, similar problems may result from lymph node dissections that result in permanent morbidity with little overall benefit to the patient.

The sentinel lymph node is theoretically the first draining lymph node that the lymphatic vessels enter after leaving an organ or area of the body. Detailed pathological examination of the sentinel lymph node predicts the spread of cancer to the other draining nodes. If the sentinel lymph node does not contain any cancerous cells, the likelihood that there is cancer in the remaining nodes is extremely small. If cancer is detected in the sentinel lymph node, then the remainder of the draining lymph nodes are excised and examined. Thus if the sentinel lymph node is negative for cancer, the standard lymph node excision, a more invasive procedure, is avoided.

Surgical excision of the sentinel lymph node requires an agent to identify the sentinel lymph node. In melanoma and breast cancer, a radioactive isotope or visible blue dye is typically injected around the primary tumor site. In breast cancer, the agent may also be injected around the areolar into the subareolar plexus of lymphatics. Depending on the type of agent used, a variable amount of time is allowed to elapse to enable the agent to enter the lymphatic system and travel to the draining lymph node. Sometimes the area of injection is manually massaged to promote uptake of the agent into the lymphatic system. If blue dye is used, a skin incision is made over the area where the sentinel lymph node is typically found and the area dissected until a blue lymph node is visually identified. If radioactive isotope is used, a gamma probe is used to identify the radioactive counts through the skin and guide the dissection. Often both the blue dye and radioactive isotope are used together to increase the sensitivity of identification.

Mattrey (US 2002/0061280 A1) and Ottoboni (WO 01/12071 A1) disclose methods of identifying the sentinel lymph node using a radiological contrast agent. Once the sentinel lymph node is identified by ultrasound imaging, computerized tomography (CT) scanning or magnetic resonance imaging (MRI), that node is excised using standard surgical technique which is an open surgical procedure performed in the operating room.

What is needed is an improved method of percutaneous excision of the sentinel lymph node using a medical device and contrast imaging to identify and guide the excision. Ideally, the percutaneous excision procedure is a minimally invasive procedure.

### SUMMARY OF THE INVENTION

A system and method for percutaneous removal of a sentinel lymph node using a radiological contrast agent for identification and guidance of the procedure are disclosed. It should be appreciated that the present invention can be implemented in numerous ways, including as a process, an apparatus, a system, a device, or a method. Several inventive embodiments of the present invention are described herein.

The process for percutaneous removal of a sentinel lymph node using a radiological contrast agent involves the use of the radiological contrast agent not only for identification of the sentinel lymph node but also for guidance of the percutaneous excision to remove the identified sentinel lymph node. The process of image guided identification and excision of the sentinel lymph node thus avoids the need for an open surgical procedure and allows for a percutaneous excision procedure.

The method generally includes injecting a radiological contrast agent detectable by an imaging modality into an area of interest, identifying a sentinel lymph node in at least one area of draining lymph nodes from the area of interest by imaging the area(s) of draining lymph nodes utilizing the imaging modality, introducing a percutaneous excision device into the area(s) of the draining lymph nodes, and excising the identified sentinel lymph node in the area(s) of draining lymph nodes using the percutaneous excision device, where the introducing and/or the excising is performed under guidance by imaging at least portions of the identified sentinel lymph node and the percutaneous excision device. The imaging modality may be ultrasound imaging, computerized tomography (CT) scanning and/or magnetic resonance imaging (MRI). A second agent may be injected with the radiological contrast agent for increased sensitivity of identification.

In another embodiment, a method for removing a sentinel lymph node generally includes injecting a radiological contrast agent and a second agent into an area of interest, imaging at least one area of draining lymph nodes from the area of interest utilizing a first imaging modality capable of detecting at least one of the radiological contrast agent and the second agent to identify a sentinel lymph node, and excising the identified sentinel lymph node in the at least one area of draining lymph nodes, where at least one of the imaging and excising includes detection utilizing the second agent to confirm identification of the sentinel lymph node.

These and other features and advantages of the present invention will be presented in more detail in the following detailed description and the accompanying figures which illustrate by way of example the principles of the invention.

### BRIEF DESCRIPTION OF THE DRAWING

The present invention will be readily understood by the following detailed description in conjunction with the accompanying drawing, wherein like reference numerals designate like structural elements.

FIG. 1 is a flowchart illustrating an exemplary process for percutaneous removal of a sentinel lymph node using a radiological contrast agent for identification and guidance of the procedure.

### DESCRIPTION OF SPECIFIC EMBODIMENTS

A system and method for percutaneous removal of a sentinel lymph node using a radiological contrast agent for identification and guidance of the procedure are disclosed. The following description is presented to enable any person skilled in the art to make and use the invention. Descriptions of specific embodiments and applications are provided only as examples and various modifications will be readily apparent to those skilled in the art. The general principles defined herein may be applied to other embodiments and applications without departing from the spirit and scope of the invention. Thus, the present invention is to be accorded the widest scope encompassing numerous alternatives, modifications and equivalents consistent with the principles and features disclosed herein. For purpose of clarity, details relating to technical material that is known in the technical fields related to the invention have not been described in detail so as not to unnecessarily obscure the present invention.

The process for percutaneous removal of a sentinel lymph node using a radiological contrast agent involves the use of the radiological contrast agent not only to facilitate the identification of the sentinel lymph node but also to facilitate guidance of the percutaneous excision to remove the identified sentinel lymph node.

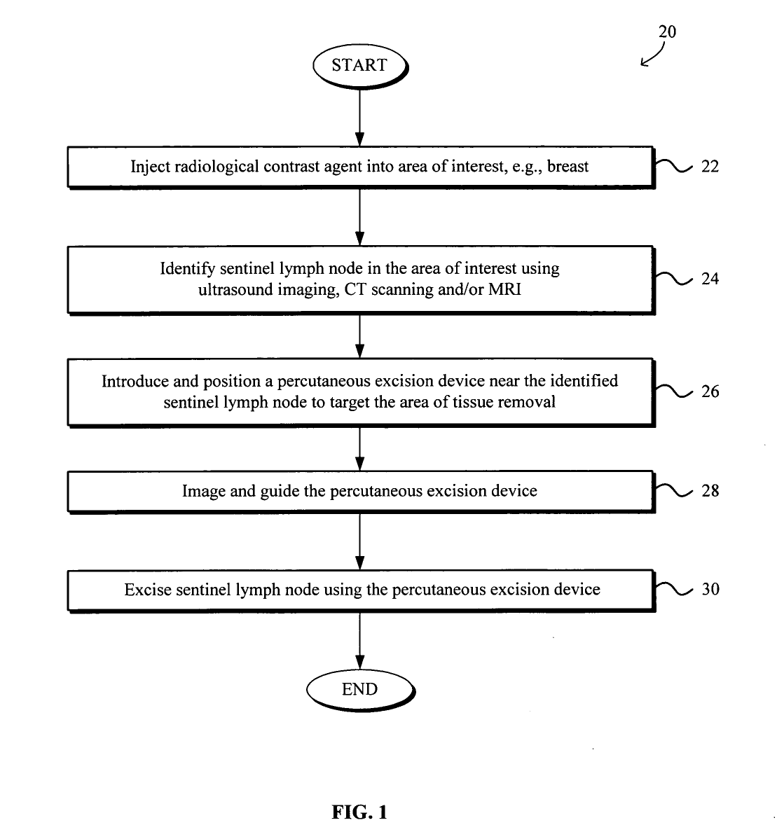

FIG. 1 is a flowchart illustrating an exemplary process 20 for percutaneous removal of a sentinel lymph node using a radiological contrast agent for identification and guidance of the procedure. At step 22, a radiological contrast agent is injected into the area of interest. In particular, radiological contrast agent particles are injected around the tumor or tumor bed and/or subcutaneously (e.g. subcutaneously around the areola of the breast) to enter the lymph vessel(s) through gaps between lymphatic endothelial cells and/or by trancellular endo- or exocytosis. Generally, smaller particles, e.g., 10 to 40 nm, are more likely to enter the lymph vessels than larger particles. Thus, the radiological contrast agent particles are preferably less than 1 μm and more preferably 40 nm or smaller.

The radiological contrast agent may be any suitable contrast agent for ultrasound imaging, CT scanning and/or MRI. Furthermore, the contrast agent can be mixed with other agents that are detectable using other detection modality or modalities such as a radioisotope detectable using a gamma counter and/or a blue dye detectable by visual inspection. The combination of a contrast agent with additional agent(s) detectable using another detection modality provides one or more additional ways of confirming/validating the target sentinel lymph node and may improve the sensitivity of the identification of the sentinel lymph node.

For breast cancer, the radiological contrast agent may be injected around the areolar into the subareolar plexus of lymphatics or around the tumor site. Depending on the type of contrast agent used, a certain amount of time may elapse to enable the contrast agent to enter the lymphatic system and travel to the draining sentinel lymph node. The area of injection may be manually massaged to promote uptake of the contrast agent into the lymphatic system.

At step 24, a sentinel lymph node is identified in the area of interest using, for example, ultrasound imaging, CT scanning and/or MRI. With ultrasound imaging, an ultrasound scanner is used and the ultrasound scanner may be configured to image radiological or ultrasound contrast agents with a specific imaging characteristic. When ultrasound is the imaging modality, ultrasound sound waves emitted by the ultrasound scanner may cause microbubble contrast agents to burst or rupture allowing the ultrasound scanner to image the acoustic signature of the bursting bubbles with greater resolution.

At step 26, a percutaneous excision device is introduced and positioned near the identified sentinel lymph node to target the area of tissue removal. At step 28, the percutaneous excision device is preferably imaged and guided, such as by using the imaging of step 24 and/or any other suitable imaging method. In particular, the percutaneous excision device may be guided so as to position the percutaneous excision device for excision of the identified sentinel lymph node. At step 30, the sentinel lymph node is excised using the percutaneous excision device. The excision step 30 is also preferably guided using, for example, the imaging of step 24 and/or any other suitable imaging method. Such image-guided identification and excision of the sentinel lymph node allows for a percutaneous excision procedure and thus helps to avoid the need for an open surgical procedure. Preferably, the process 20 uses a minimally invasive percutaneous excision device, such as one disclosed in U.S. patent application Ser. No. 10/087,412, entitled “Devices and Methods for Tissue Severing and Removal” and filed on Mar. 12, 2002, the entirety of which is incorporated herein. Any other suitable percutaneous excision devices may be similarly used for percutaneous removal of the sentinel lymph node. As used herein, percutaneous excision devices refer to excision devices that require an incision that is generally only large enough to insert the excision device.

For breast cancer using a combination of ultrasound contrast agent and at least one other agent detectable by a different detection modality, e.g., a blue dye and/or radioisotope, the agents can be injected together in the periareolar lymphatic plexis and/or around the cancer. The breast is preferably manually massaged to facilitate uptake of the agents into the lymphatic system. One or more areas of the draining lymph nodes (e.g. ipsilateral axilla, ipsilateral internal mammary lymph nodes) are scanned with, for example, a standard ultrasound probe to locate the sentinel lymph node that has taken up the ultrasound contrast agent. After appropriate anesthetic is delivered, e.g. local anesthesia, a small incision is made and a percutaneous, e.g., minimally invasive, excision device is inserted into one or more areas of the draining lymph nodes through the incision. Under ultrasound guidance, the percutaneous excision device can be directed adjacent to the target sentinel lymph node and activated to separate the sentinel lymph node from the surrounding tissue and removed. Confirmation that the tissue removed is the target sentinel lymph node may be made by a visual inspection of the removed sentinel lymph node where a blue dye was injected with the ultrasound contrast agent, i.e., the sentinel lymph node appears blue, and/or by taking a count with a gamma probe placed on the removed sentinel lymph node where a radioisotope was injected with the ultrasound contrast agent and/or by ultrasound scanning of the tissue removed to identify ultrasound contrast agent in the tissue removed. The process using the combination of ultrasound contrast agent and another agent may be performed using an open surgical excision of the sentinel lymph node or using a percutaneous excision device.

In the case of using a combination of ultrasound contrast agent and a radioisotope detectable by a gamma probe, such as in the case of breast cancer, confirmation of the sentinel lymph node using the gamma probe can be alternatively or additionally be made prior to insertion of the excision device at step 26 by placing the gamma probe on the skin over the sentinel lymph node.

While the exemplary embodiments of the present invention are described and illustrated herein, it will be appreciated that they are merely illustrative and that modifications can be made to these embodiments without departing from the spirit and scope of the invention. Thus, the scope of the invention is intended to be defined only in terms of the following claims as may be amended, with each claim being expressly incorporated into this Description of Specific Embodiments as an embodiment of the invention.

What is claimed is:

1. A method for removing a sentinel lymph node, comprising the steps of:
injecting a radiological contrast agent detectable by an imaging modality into an area of interest;
identifying a sentinel lymph node in at least one area of draining lymph nodes that receive lymphatic fluid from the area of interest by imaging the at least one area of draining lymph nodes utilizing the imaging modality;
introducing a percutaneous excision device into the at least one area of draining lymph nodes; and
excising the identified sentinel lymph node in the at least one area of draining lymph nodes using the percutaneous excision device, where at least one of the introducing and the excising is performed under guidance by imaging at least portions of the identified sentinel lymph node and the excision device.

2. The method of claim 1, in which the imaging modality is selected from the group consisting of ultrasound imaging, computerized tomography (CT) scanning and magnetic resonance imaging (MRI).

3. The method of claim 1, in which during the imaging, at least portions of the identified sentinel lymph node and the percutaneous excision device are imaged utilizing the imaging modality capable of detecting the injected radiological contrast agent.

4. The method of claim 1, in which the step of introducing the percutaneous excision device into the area of draining lymph nodes is performed under guidance by imaging at least portions of the identified sentinel lymph node and the percutaneous excision device utilizing the imaging modality and in which the step of excising the identified sentinel lymph node using the percutaneous excision device is performed under guidance by imaging at least portions of the identified sentinel lymph node and the percutaneous excision device utilizing the imaging modality.

5. The method of claim 1, in which the injecting includes injecting the radiological contrast agent and at least one other agent to facilitate identification of the sentinel lymph node.

6. The method of claim 5, in which the other agent is selected from the group consisting of a radioisotope and a blue dye.

7. The method of claim 1, in which the step of injecting further includes injection of a second agent in combination with the radiological contrast agent, further comprising the step of confirming the identification of the target sentinel lymph node after the excision using a detection modality different from the imaging modality.

8. The method of claim 7, in which the second agent is a radioisotope and the detection modality is a gamma counter.

9. The method of claim 7, in which the second agent is a blue dye and the detection modality is visual inspection.

10. The method of claim 1, in which the area of interest is human breast.

11. A method, comprising:
injecting a radiological contrast agent and a second agent into an area of interest;
imaging at least one area of draining lymph nodes that receive lymphatic fluid from the area of interest utilizing a first imaging modality capable of detecting the radiological contrast agent; and
excising, using a percutaneous excision device, the identified sentinel lymph node in the at least one area of draining lymph nodes, where at least one of the imaging and excising includes detection of the sentinel lymph node utilizing the second agent to confirm identification of the sentinel lymph node.

12. The method of claim 11, in which the first imaging modality is capable of detecting the radiological contrast agent and is selected from the group consisting of ultrasound imaging, computerized tomography (CT) scanning and magnetic resonance imaging (MRI).

13. The method of claim 11, further comprising introducing a percutaneous excision device into the at least one area of draining lymph nodes and in which the excision is performed utilizing the percutaneous excision device.

14. The method of claim 13, in which excising the identified sentinel lymph node using the percutaneous excision device is performed under guidance by imaging at least portions of the identified sentinel lymph node and the percutaneous excision device utilizing at least one of the first imaging modality and detection utilizing the second agent.

15. The method of claim 11, in which the second agent is selected from the group consisting of a radioisotope and a blue dye.

16. The method of claim 11, in which the first imaging modality is ultrasound and in which the second agent is a radioisotope and the detection utilizing the second agent utilizes a gamma counter.

17. The method of claim 11, in which the second agent is a blue dye and the detection utilizing the second agent is via visual inspection, and includes visually examining the excised sentinel lymph node to confirm identification of the sentinel lymph node upon appearance of the blue dye.

18. The method of claim 11, in which the area of interest is human breast.
